(19) United States
(12) Patent Application Publication
(10) Pub. No.: US 2009/0216336 A1
(43) Pub. Date: Aug. 27, 2009

(54) BIORESORBABLE, MINERALISED MATERIAL FOR FILLING OSSEOUS DEFECTS

(76) Inventors: Marco Springer, Esslingen (DE); Arne Briest, Karlsruhe (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/719,864

(22) PCT Filed: Nov. 22, 2005
(86) PCT No.: PCT/EP2005/012453

§ 371 Date: Oct. 7, 2008

(30) Foreign Application Priority Data
Nov. 23, 2004 (DE) 10 2004 058 479.6
Apr. 4, 2005 (DE) 10 2005 016 443.9
Jul. 13, 2005 (DE) 10 2005 034 421.6

(51) Int. Cl. A61F 2/28; C07K 14/78; A61K 47/42; A61P 19/00; B29C 35/16

(52) U.S. Cl. 623/23.61; 514/773; 264/28

(57) ABSTRACT

A bioresorbable and mineralized material for filling osseous defects includes a native and/or renatured collagen matrix of congregated collagen chains, in which essentially only a surface of the congregated collagen chains is mineralized. A shaped article for filling osseous defects includes such a material. A method for producing a shaped article for filling osseous defects from a liquid medium, includes the steps of deposition of a mineral substance, preferably from its dissolved ionogenic components, on a collagen matrix, precipitation of the mineralized collagen matrix, separation of the mineralized collagen matrix, transfer of the mineralized collagen matrix to a suspension, transfer of the suspension to a mold defining the shaped article, and freeze-drying of the suspension in the mold.

The invention relates in the first instance to a bioresorbable and mineralized material for filling osseous defects.

About 70% of the natural bone is composed of inorganic components, in particular of hydroxyapatite, about 20% is composed of organic components, in particular of type I collagen, which are produced by bone-forming cells, called osteoblasts, and secreted and deposited, and about 10% is composed of water. There is also a small proportion of so-called non-collagen proteins, in particular osteocalcin, osteonectin and osteopontin, cytokines, growth factors, proteoglycans and fats.

Natural bone is subject to a constant process of break-up and regeneration, referred to as remodelling. The formation of new bone, called osteogenesis, is induced by the bone itself, in the same way as for healing a fracture. The bone thus possesses all the information needed to form functional bone. However, natural limits may be imposed on bone regeneration by the size of a defect and by many different functional disturbances, for example disorders of the metabolism.

The treatment of osseous defects has an important role in medical care, especially in orthopedics, emergency surgery, surgery of the jaws and teeth, and surgery of the hand. Oseeous defects may have various causes. For example, they may be caused by cysts, atrophy, tumors, etc. Complicated comminuted fractures, malformations, and loosening of implants place the greatest demands on therapy. Consequently, the focus of treatment lies increasingly in reconstructing the defect by suitable filling materials.

A great many different materials are available for filling osseous defects, in particular ones that are not bioresorbable or are poorly bioresorbable, and ones that are resorbed in the body. The first ones comprise, for example, porous calcium phosphate ceramics. Although these have sufficient osteoconductive properties, i.e. load-bearing properties on account of the presence of a structuring matrix, they nevertheless have the disadvantage that they remain within the body, and the new bone only grows through them.

The bioresorbable bone substitute materials are often synthetic polymers, in particular polylactide or polyglycolide. These are broken up in vivo by hydrolysis, which, depending on what polymer is used, can lead to the release of acid break-down products, for example lactic acid or glycolic acid, and thus to local hyperacidity in the body.

The development of bioresorbable bone substitute materials thus led to fillers that are composed predominantly of natural bone tissue, in particular of animal origin, preferably of equine, porcine or bovine origin. These materials are often demineralized bone materials in which the inorganic portion (minerals) has been almost completely removed, except for traces. The remaining organic matrix, which is composed mainly of collagen, is very similar to natural bone in terms of its composition and structure. This has the effect that the demineralized bone material has stabilizing or osteoconductive properties. An example of one such biomatrix is the lyophilized, renatured collagen extract sold commerically under the name Colloss® by the applicant and obtained from bovine bone. Colloss® supports the process of bone growth and formation of new bone by virtue of its structural biocompatibility. However, in cases where quite large osseous defects are to be treated, the load-bearing capacities of these materials may not be sufficient. In particular, it is often necessary, especially in the initial stages of treatment, to additionally stabilize the affected area of the body by using metals, for example in the form of plates or intramedullary nails.

A highly promising approach to increasing the stability of bone substitute materials based on collagen is that of mineralization of collagen (M. Gelinsky, U. König, A. Sewing, W. Pompe: Porous scaffolds of mineralized collagen—a biomimetic bone substitute material, published in German in Mat.—Wiss und Werkstofftech., 2004, 35, No. 4). However, this approach is carried out under denaturing conditions. By means of the denaturing of the collagen matrix, particularly as a result of acid treatment, the osteoinductive properties of the bone substitute material may be impaired, which ought to be avoided as far as possible in respect of the formation of new endogenous bone or cartilage tissue.

The object of the invention is therefore to make available an osteoinductive material for filling osseous defects, which material corresponds substantially to the natural composition of bone. Compared to conventional bone substitute materials based on collagen, this material is intended to have much improved osteoconductive properties, that is to say an improved load-bearing function and/or volume-stabilizing function during the reformation of endogenous bone.

This object is achieved by a material as described in claim 1. Preferred embodiments of this material are set forth in dependent claims 2 to 18. Shaped articles, which are at least partially made of the material according to the invention, are set forth in claims 19 to 21. Claims 22 to 28 relate to a suitable method for producing such a shaped article for filling osseous defects. The wording of all the claims is incorporated by reference in the content of the description.

The material according to the invention for filling osseous defects is a bioresorbable and mineralized material which comprises, among other things, a collagen matrix of congregated or aggregated collagen chains and molecules, it being essentially only the surface of the congregated or individually congregating collagen chains that is mineralized, i.e. encapsulated by crystallites of a mineral substance. Preferably, the collagen is mainly type I collagen.

The material to be mineralized can be a native collagen matrix with triple-helix collagen chains (individual collagen chains are congrated into a triple helix). This is particularly preferable, since the resulting network of collagen fibers and/or fibrils is present in its natural state and does not have to be brought into an artificial spatial arrangement. The result of this is that the organic matrix of collagen, left in the natural state, already has osteoconductive or load-bearing and/or volume-stabilizing properties. The native collagen matrix, in particular a matrix of type I collagen, is preferably mineralized both inside the matrix and also on its surface. This contributes to additionally increasing the osteoconductive properties of the collagen matrix.

In a particularly preferred embodiment, the material according to the invention is an originally denatured and subsequently renatured collagen matrix, the collagen chains being congregated to form a superstructure, preferably a net-like structure. Such a collagen matrix is advantageously the material called Colloss®, preferably Colloss® E, from the applicant.

Provision can also be made, according to the invention, for some of the collagen chains to be present in a triple helix structure, such that the renatured collagen matrix, in particular Colloss®, preferably Colloss® E, has both areas with a triple-helix structure and also with overstructures, in particular net-like structures.

The collagen matrix preferably contains both native collagen and also renatured collagen. The native collagen is preferably pure collagen, in particular type I collagen, which is produced for example from tendons or skin. By virtue of its aforementioned osteoconductive properties, the native collagen serves as a stabilizing component for the structure of the renatured collagen matrix, preferably of Colloss®. In a particularly preferred embodiment, the renatured collagen matrix is Colloss® E.

In a particularly preferred embodiment, the material according to the invention has osteoinductive properties (properties that induce bone growth). Preferably, the material according to the invention itself already has osteoinductive properties because of the presence of a native or renatured collagen matrix. However, it may also be preferable to achieve the osteoinductive properties of the material by doping with corresponding osteoinductive active substances.

The material according to the invention preferably comprises at least one active substance, in particular an active substance that activates and/or stimulates osteogenesis. This may be particularly preferable in order to strengthen the osteoinductive properties of the mineralized material. The active substance is preferably of native origin. Such an active substance is understood to be a native active substance, or an active substance which is present in its natural structure (configuration) and which is present in a substantially native or renatured collagen matrix. Provision is made, according to the invention, for the active substance to be present inside the collagen matrix and/or on the latter's surface. The material Colloss® from the applicant is a collagen matrix extract of bovine origin, which comprises at least one native active substance, preferably several of the active substances described below, particularly in the form of a complex. The material Colloss®° E from the applicant is a collagen matrix extract of equine origin, which comprises at least one native active substance, preferably several of the active substances described below, particularly in the form of a complex. The collagen extract of equine origin (Colloss® E) is also commercially available from the applicant and is especially preferred because of the particularly low probability of transmission of pathogens.

The material according to the invention advantageously contains so-called recruiting factors, in particular chemotactic agents (chemotaxins), for example leukotrienes, which specifically cause cells of the body, preferably mesenchymal stem cells, cartilage precursor cells, fibroblasts and/or thrombocytes, to penetrate the bioresorbable and mineralized material.

It may also be preferable for the filling material according to the invention to contain so-called adhesion factors for immobilizing the incoming cells on or in the mineralized material. The material according to the invention is advantageously distinguished by the fact that it in particular contains cytotactin, tenascin, laminin and/or fibronectin as adhesins.

In a particularly preferred embodiment of the invention, the material according to the invention comprises growth and/or maturation factors for the proliferation and differentiation of the incoming cells, in particular cytokines. The growth factors are preferably bone-growth factors, for example BMP (bovine morphogenetic protein), in particular BMP-II, BMP-VII and/or BMP-IV, and IGF (insulin-like growth factor), in particular IGF-I, and TGF (transforming growth factor), in particular TGF-βI. The material according to the invention preferably contains FGF (fibroblast growth factor) and PDG (platelet-derived growth factor) as additional growth factors.

The bioresorbable and mineralized material can advantageously comprise a combination of the active substances described above. This may be particularly preferable in order to achieve superadditive or synergistic effects in the reconstruction of a bone defect by endogenous material. According to the invention, provision is also made for the active substances, in particular the aforementioned active substances, to partially overlap in terms of their function and mode of action. In this way, the failure of one or more active substances can advantageously be compensated for, without leading to a deterioration, particularly a reduction, in the successful outcome, preferably of the osteoinductive properties. As regards the above-described examples of native active substances, reference is made to EP 0 500 556 B1.

In a particularly preferred embodiment, the material according to the invention comprises an active substance complex, preferably an active substance complex that activates and/or stimulates osteogenesis The materials Colloss® and Colloss® E from the applicant are each collagen extracts that have an osteoinductive active substance complex. For other properties of the active substance complex, particularly in terms of its composition, reference is made to the above description.

It may additionally be preferable for the bioresorbable and mineralized material to comprise an antimicrobiotic substance. The antimicrobiotic substance is advantageously an antibiotic, which prevents or suppresses possible immune defense reactions in the body.

It is also possible for the material to be equipped with a cytostatic agent. This is particularly advantageous in cases of cancerous changes in the area of the bone defect and/or of the surrounding tissue. However, the use of other active substances, for example of antibodies, particularly for therapeutic reasons, may also be useful in this context. In a development of the invention, the various active substances that have been mentioned by way of example and other active substances can be combined with one another, in order to achieve particularly advantageous effects, in particular superadditive or synergistic effects. As regards compensating for failure of an active substance, reference is made to what has already been stated above.

In another particularly preferred embodiment of the invention, the material according to the invention is mineralized, on the surface of the congregated collagen chains, with calcium phosphate and/or calcium carbonate and/or hydroxyapatite. Calcium phosphate in the form of the hydroxyapatite (calcium phosphate hydroxide) is the main constituent of the mineral bone substance, which makes up 50% of the bone volume. Together with calcium carbonate, it essentially determines the hardness of the natural bone. Both calcium phosphate and calcium carbonate are therefore preferably suitable for increasing the osteoconductive properties and load-bearing properties of a collagen matrix. The deposition of calcium phosphate and/or calcium carbonate on the surface of the congregated collagen chains leads to a subsequent strengthening of the collagen matrix.

In a development of the invention, the material according to the invention has pores, preferably interconnecting pores. The latter means that the pore structure is optimized not only between the individual particles of the material according to the invention, but also within the particles. In addition, the interconnecting porosity is also particularly advantageously accessible to cells. It may also be preferable for the active substances described here, but also for all other active substances, to be presented on the inner and/or outer surface of the material according to the invention in order to make these substances available to the body, in particular to endogenous cells.

In a preferred embodiment, the pores of the material according to the invention have pore diameters of at least 100 μm, in particular pore diameters of between 100 and 300 μm, preferably of ca. 200 μm. This is particularly advantageous, because pore diameters of at least 100 μm are necessary to permit infiltration of bone cells into the porous material according to the invention. This promotes the integration of the material according to the invention in the body and also its break-up and resorption.

In a development of the invention, the material is sterilized or presented in a sterilized form. Many conventional methods may be considered for sterilizing the material according to the invention. The bioresorbable and mineralized material is preferably sterilized by radioactive irradiation, preferably by gamma irradiation. It is also preferable for the material according to the invention to be treated with ethylene oxide gas.

In another development of the invention, the material according to the invention is treated and/or produced aseptically, for example by use of sterile-filtered antibiotic solutions, preferably a sterile-filtered gentamycin solution. This may be particularly preferred, because it avoids the possible problems associated with sterilization, for example a deleterious effect on the added active substances and/or on the osteoinductive properties of the material.

According to the invention, the material is presented in packaged form, particularly in a sterile packaged form. In such a package, the material can be stored for quite a long period of time, in particular for a period of several months, without any deterioration in the effect of the bioresorbable and mineralized material, in particular without any deterioration in its osteoinductive properties. At the time of use, the material can easily be removed from the package and applied. To achieve the sterility of the material according to the invention, it may also be preferable to sterilize the material inside the package, for example by irradiation or by ethylene oxide gas. Plastics are particularly suitable as packaging materials.

In another preferred embodiment of the invention, the material additionally contains a mixture of a predominantly native and/or renatured collagen matrix and hyaluronic acid, the mixture being essentially spatially separate from the mineralized collagen matrix. The mixture preferably contains Colloss® as additional collagen component. Particularly preferably, the mixture contains Colloss® E as additional collagen component. The hyaluronic acid, a constituent of the so-called synovial fluid, acts as a lubricant for the joints and is responsible for supplying the cartilage with nutrients through diffusion. Preferably, the composition of mineralized collagen matrix and of the mixture of a predominantly native collagen matrix and hyaluronic acid, preferably also mineralized together, is suitable for the treatment of chondral defects, in particular of osteochondral defects.

In the last-mentioned embodiments, it is preferable that the spatially separated parts of this composition are physically connected to one another, in particular by a chemical cross-linking. The composition is preferably cross-linked with a carbodiimide, in particular with N-(dimethylaminopropyl)-N′-ethyl carbodiimide (EDC). The chemical cross-linking leads to further stabilizing of this material according to the invention.

The invention also relates to a shaped article for filling osseous defects, this shaped article comprising a material according to the invention with the above-described properties, and preferably consisting of this material. Shaped articles are thus to be understood as aggregates of mineralized, substantially native and/or renatured collagen matrix, which may optionally contain a further component, for example a mixture of predominantly native and/or renatured collagen matrix and hyaluronic acid. These shaped articles are preferably presented as three-dimensional shaped articles, in particular as bars, rings or cylinders, preferably as hollow cylinders. It is also possible, however, for the shaped articles to be presented as cubes, disks or the like. Shaped articles of any desired forms can be produced.

According to a particularly preferred embodiment, the shaped article is a hollow cylinder whose hollow space comprises a non-mineralized substance, in particular collagen, preferably type I collagen. The hollow space of the hollow cylinder can be at least partially filled, preferably completely filled, with the non-mineralized substance, in particular collagen. In addition, or as an alternative to this, it is possible, according to the invention, for the inner surface of the hollow cylinder to be at least partially coated, preferably completely coated, with the non-mineralized substance, in particular collagen. The collagen can in particular be a collagen of the kind described in the preceding embodiments, particular preference being given to a collagen, in particular Colloss®, preferably Colloss® E, comprising at least one active substance, preferably several active substances. As regards the active substances, reference is made to the above description, with particular preference being given to the active substances that activate and/or stimulate osteogenesis. The hollow cylinder can be open at one or both ends, i.e. can have an opening with a defined diameter (internal diameter of the hollow cylinder). The hollow cylinder is preferably open at both ends. The open and in particular porous structure (see above description) of the hollow cylinder permits the passage of body cells, in particular of bone cells or their precursor cells, into the hollow space of the cylinder.

The hollow cylinder is further characterized in particular in that it has an external diameter of 10 to 20 mm, preferably of 10 to 15 mm, and an internal diameter of 3 to 12 mm. The hollow cylinder can preferably have a length of 20 to 60 mm, in particular of 30 to 50 mm, preferably of ca. 40 mm. The hollow cylinder preferably has an external diameter of 10 to 15 mm, an internal diameter of 3 to 12 mm and a length of ca. 40 mm, because the resulting overall volume of the hollow cylinder (including the volume of the hollow space) corresponds approximately to the volume or space between two human vertebral bones. The hollow cylinder is therefore suitable in particular for stabilization of preferably human vertebral bones.

In a preferred embodiment of the invention, the shaped articles are further stabilized by a chemical cross-linking, in particular with a carbodiimide, preferably with N-(dimethylaminopropyl)-N′-ethyl carbodiimide (EDC). The shaped articles can be further stabilized by introduction of a binder and/or salt. The shaped articles thus obtained retain their basic shape after wetting with liquids. Preferably, they can more or less resume their original shape after compression, that is to say they have elastic properties.

Finally, the invention relates to a method for producing a shaped article from a liquid medium, preferably for filling osseous defects, i.e. essentially from a material having the properties described above, said method comprising the following steps:
deposition of a mineral substance, preferably from its dissolved ionogenic components, onto a collagen matrix,
precipitation of the mineralized collagen matrix,
separation of the mineralized collagen matrix,
transfer of the mineralized collagen matrix to a suspension,
transfer of the suspension to a mold defining the shaped article,
freeze-drying of the suspension in the mold.

Solutions, in particular aqueous solutions, of the ionogenic components that form the mineral substance to be deposited are preferably added to a collagen suspension. The suspension preferably contains native or renatured collagen, with particular preference being given to Colloss® or Colloss® E, in particular to Colloss® E. It is also possible that the minerals are a waste product of the Colloss® or Colloss® E production process and in particular are added again to a Colloss® or Colloss® E suspension.

It is particularly preferable that the deposition of the mineral substance is done essentially only on the surface of the collagen matrix, preferably on the surface of the congregated collagen chains. The deposition of the mineral substance is carried out in a neutral pH range, in particular in a pH range of between 6 and 8, preferably at a pH value of 7, in order to achieve the selective surface deposition on the collagen matrix.

The precipitation of the mineralized collagen matrix is preferably achieved by exceeding the solubility product of the mineral substance in solution. This can be observed externally from the formation of a deposit that mainly contains mineralized collagen. The test parameters, in particular the concentration of the mineral substance and the pH value, must be set such that a synchronous assembling of the collagen and of the mineral substance takes place.

The precipitation of the collagen matrix is advantageously carried out with calcium phosphate and/or calcium carbonate and/or hydroxyapatite as mineral substance.

According to the invention, it may be preferable that, before precipitation of the mineralized collagen matrix, preferably before deposition of the mineral substance on the collagen matrix, native pure collagen, in particular type I collagen, is added. The native pure collagen is advantageously added in the form of a suspension. As has already been mentioned, the added collagen serves as a stabilizing component and leads to a strengthening of the structure, in particular to an increase in the osteoconductive properties, of the collagen matrix, in particular of Colloss®, preferably of Colloss® E. A subsequent mineralization (deposition of the mineral substance on the collagen matrix and precipitation of the mineralized collagen matrix) preferably has the result that the collagen matrix to be stabilized, in particular Colloss®, preferably Colloss® E, and the added native pure collagen are together covered by a layer of the mineral substance.

In another preferred embodiment of the invention, the mineralization of the collagen matrix, in particular of Colloss®, preferably of Colloss® E, and that of the native pure collagen, in particular type I collagen, are carried out separately. The resulting suspensions of mineralized collagen matrix, in particular of Colloss®, preferably of Colloss® E, and of mineralized native pure collagen are mixed and further processed according to one of the following steps in particular.

In a development of the method according to the invention, the mineralized collagen, after precipitation, is separated from the solution, for example by centrifuging.

According to the invention, the mineralized collagen separated from the solution is transferred into a preferably pourable and homogeneous suspension. This suspension can be poured into suitable mold cavities, in particular into the cavities of a so-called well plate, for example a 24-well or 96-well plate, in particular made of polystyrene. Depending on the shapes of the cavities, different three-dimensional and preferably porous shaped articles can be produced by freeze-drying, in particular bars, rings, cylinders, preferably hollow cylinders, disks, cubes or the like, with the production of hollow cylinders being particularly preferred. By selecting the speed and temperature of the freeze-drying process, it is possible to particularly advantageously influence the pore size. The volumes of the suspension transferred into the mold cavities of a well plate are preferably frozen slowly at temperatures of between −10 and −40° C. and freeze-dried in an oil pump vacuum.

The shaped articles thus obtained are preferably cross-linked by a chemical cross-linking, in particular with a carbodiimide, preferably with N-(dimethylaminopropyl)-N′-ethyl carbodiimide (EDC). The shaped articles thus produced advantageously retain their basic shape after wetting with liquids, and they also more or less resume their original shape following compression.

In another preferred embodiment of the invention, a shaped article composed of native pure collagen, in particular of type I collagen, can be doped or modified with the collagen matrix, in particular with Colloss®, preferably with Colloss® E. The collagen matrix, in particular Colloss®, preferably Colloss® E, can be applied in the form of a suspension or of a gel to the shaped article, in particular a collagen sponge, and preferably connected to the latter by freeze-drying.

The present material according to the invention for filling osseous defects is a bone substitute material that combines osteoinductive properties with osteoconductive properties in a particularly advantageous way. This is achieved preferably by subsequent strengthening of the collagen matrix by deposition of a mineral substance on the surface of the collagen fibrils. The mechanical strengthening of the essentially native or renatured collagen matrix results in osteoinductive properties of the material, which can be strengthened specifically by addition of further substances that stimulate induction of osteogenesis. The material according to the invention is therefore very particularly suitable for the treatment of large and complicated bone defects in which sufficient stability of the bone substitute material and a rapid infiltration of bone cells into the bone substitute material are needed.

Further features and advantages of the invention will become clear from the following description of a preferred embodiment given by way of an example and in conjunction with the dependent claims. The individual features can appear singly or in combination with one another.

### EXAMPLE 1

1 g of collagen suspension (Colloss® from the applicant) is made up with water to 1 liter, after which the following solutions are added in sequence and with vigorous stirring: 500 ml of distilled water, 180 ml of 0.1 mol calcium chloride solution, 116 ml of 2 mol sodium chloride solution, 168 ml of 0.5 mol tris buffer (pH=7.52) and 22.6 ml of 0.5 mol phosphate buffer after Sörensen (pH=7.40). The mixture is made up with distilled water to 2 liters and is left to stand for 12 hours at 35° C. in water. The resulting colorless, gelatinous precipitate is centrifuged off and then agitated again with sufficient mother liquor to form a pourable and homogeneous suspension. To produce the cylindrical standard specimens, ca. 2.5 ml of the suspension are in each case poured into the cavities of a 24-well cell culture plate made of polystyrene and are slowly frozen at −25° C. The frozen material is freeze-dried in the oil pump vacuum and then chemically cross-linked for 1 hour in a 1%-strength solution of N-(dimethylaminopropyl)-N′-ethyl carbodiimide hydrochloride (EDC, from Aldrich) in 80%-strength ethanol. The porous scaffolds are thereafter washed thoroughly with distilled water, then with a 1%-strength glycine solution, and finally once again with water, after which they are freeze-dried again. For the purpose of sterility, the scaffolds are either produced aseptically, sterilized by gamma irradiation or treated with ethylene oxide gas. In the product thus obtained, the individual collagen chains that form the net-like structure are in each case encapsulated by calcium phosphate crystallites.

### EXAMPLE 2

1 g of collagen suspension (Colloss® E from the applicant) is made up with water to 1 liter, after which the following solutions are added in sequence and with vigorous stirring: 500 ml of distilled water, 180 ml of 0.1 mol calcium chloride solution, 116 ml of 2 mol sodium chloride solution, 168 ml of 0.5 mol tris buffer (pH=7.52) and 22.6 ml of 0.5 mol phosphate buffer after Sörensen (pH=7.40). The mixture is made up with distilled water to 2 liters and is left to stand for 12 hours at 35° C. in water. The resulting colorless, gelatinous precipitate is centrifuged off and then agitated again with sufficient mother liquor to form a pourable and homogeneous suspension. To produce the cylindrical standard specimens, ca. 2.5 ml of the suspension are in each case poured into the cavities of a 24-well cell culture plate made of polystyrene and are slowly frozen at −25° C. The frozen material is freeze-dried in the oil pump vacuum and then chemically cross-linked for 1 hour in a 1%-strength solution of N-(dimethylaminopropyl)-N′-ethyl carbodiimide hydrochloride (EDC, from Aldrich) in 80%-strength ethanol. The porous scaffolds are thereafter washed thoroughly with distilled water, then with a 1%-strength glycine solution, and finally once again with water, after which they are freeze-dried again. For the purpose of sterility, the scaffolds are either produced aseptically, sterilized by gamma irradiation or treated with ethylene oxide gas. In the product thus obtained, the individual collagen chains that form the net-like structure are in each case encapsulated by calcium phosphate crystallites.

1. A bioresorbable and mineralized material for filling osseous defects, comprising a collagen matrix of congregated collagen chains, wherein essentially only a surface of the congregated collagen chains mineralized.

2. The material as claimed in claim 1, wherein the collagen matrix contains native and/or renatured collagen.

3. (canceled)

4. (canceled)

5. The material as claimed in claim 1, wherein the collagen chains are congregated to form a net-like structure.

6. The material as claimed in claim 1, wherein the material has osteoinductive properties.

7. The material as claimed in claim 1, wherein the material comprises at least one active substance.

8. The material as claimed in claim 7, wherein the active substance is of native origin.

9. The material as claimed in claim 8, wherein the material involves a recruiting and/or adhesion and/or growth and/or maturation factor.

10. (canceled)

11. The material as claimed claim 1, wherein the surface of the congregated collagen chains is mineralized with calcium phosphate and/or calcium carbonate and/or hydroxyapatite.

12. (canceled)

13. The material as claimed in claim 12, wherein the material has pores having pore diameters of at least 100 μm.

14. (canceled)

15. (canceled)

16. The material as claimed in claim 1, wherein the material contains a mixture of a predominantly native and/or renatured collagen matrix and hyaluronic acid, the mixture being essentially spatially separate from the pure mineralized collagen matrix.

17. The material as claimed in claim 16, wherein the mixture and the mineralized collagen matrix are connected to one another by a chemical cross-linking.

18. (canceled)

19. A shaped article for filling osseous defects, comprising a material as claimed in claim 1.

20. The shaped article as claimed in claim 19, wherein the material is presented as a bar, ring cylinder or hollow cylinder.

21. The shaped article as claimed in claim 19, wherein the material is presented as a hollow cylinder having a hollow space that comprises a non-mineralized substance.

22. A method for producing a shaped article for filling osseous defects from a liquid medium, from essentially a material as claimed in claim 1, said method comprising the following steps:
deposition of a mineral substance on a collagen matrix,
precipitation of the mineralized collagen matrix,
separation of the mineralized collagen matrix,
transfer of the mineralized collagen matrix to a suspension,
transfer of the suspension to a mold defining the shaped article, and
freeze-drying of the suspension in the mold.

23. The method as claimed in claim 22, wherein the deposition of the mineral substance is done essentially only on the surface of the collagen matrix.

24. The method as claimed in claim 22, wherein the precipitation of the mineralized collagen matrix is initiated by exceeding the solubility product of the mineral substance.

25. The method as claimed in claim 22, wherein the precipitation of the collagen matrix is carried out with calcium phosphate and/or calcium carbonate and/or hydroxyapatite as mineral substance.

26. The method as claimed in claim 22, wherein, before precipitation of the mineralized collagen matrix, native pure collagen is added.

27. The method as claimed in claim 22, wherein a suspension of the mineralized collagen matrix and a suspension of the mineralized native pure collagen are mixed and further processed.

28. The method as claimed in claim 22, wherein a shaped article composed of native pure collagen is modified with the collagen matrix.

29. The material as claimed in claim 1, wherein the collagen matrix is a lyophilized, renatured collagen extract.

30. The material as claimed in claim 1, wherein the collagen matrix is a collagen matrix extract of bovine origin, which comprises at least one native active substance.

31. The material as claimed in claim 1, wherein the collagen matrix is a collagen matrix extract of equine origin, which comprises at least one native active substance.
